(19) United States
(12) Patent Application Publication
(10) Pub. No.: US 2002/0198228 A1
(43) Pub. Date: Dec. 26, 2002

(54) Composition and method for the treatment of respiratory desease

(76) Inventor: Sita R. Kaura, West Bloomfield, MI (US)

Correspondence Address: POWELL, GOLDSTEIN, FRAZER & MURPHY LLP, P.O. BOX 97223, WASHINGTON, DC, 20090-7223, US

(21) Appl. No.: 09/825,258

(22) Filed: Apr. 3, 2001

(51) Int. Cl. A61K 31/473

(52) U.S. Cl. 514/290

(57) ABSTRACT

A method and composition for the treatment of respiratory disease, particularly bronchial asthma, is provided. The method and composition is provided in the form of an oral medication and includes a combination of known medications so as to avoid unnecessary undesirable reactions. The treatment composition and regimen includes a pair of compatible receptor antagonists and an adrenergic bronchodilator. The pair of compatible receptor antagonists include a leukotriene receptor antagonist and a histamine H₁,-receptor antagonist. The adrenergic bronchodilator is a beta₂-adrenergic bronchodilator.

### BACKGROUND OF THE INVENTION

1. Technical Field

The present invention relates to a composition and method for the treatment of respiratory disease. More particularly, the present invention provides such a composition and method which is directed to the treatment and prevention of chronic bronchial asthma.

2. Summary of Related Art

Broadly speaking and true to its Greek root, “asthma” at one 10 time referred to a general difficulty in breathing, and still today is regarded generally as a chronic inflammatory disease of the airways. General manifestations of asthma include shortness of breath, coughing, wheezing, and chest tightness, and all of these symptoms usually worsen at night. Chronic asthma is truly an 15 around-the-clock ailment, resulting in sleep disturbance and nighttime awakening and early morning asthma and daytime symptoms. Exercise (particularly in cold air) and stressful conditions exacerbate the symptoms, which may also be worsened by taking aspirin or other anti-inflammatory medications. Naturally, 20 exposure to allergens or occupational exposures initiate asthmatic characteristics.

The word “asthma” is generic and appears in many specific other, less common forms, including atopic asthma, cardiac asthma, dust asthma, food asthma, extrinsic and intrinsic asthma, and so-called summer asthma.

Bronchial asthma is surprisingly wide-spread and affects nearly 20 million Americans, of which about 25 percent are children. While ordinarily not life-threatening, this condition in its extreme form causes over five thousand deaths annually. Bronchial asthma is defined by a condition of the lungs in which the airways are narrowed. This narrowing is ordinarily widespread and is caused by hypertrophy and contraction of smooth muscle in the walls of the bronchi and the bronchioles (bronchoconstriction brought about by bronchospasms), thickening of subepithelial basal membrane, sub-mucosal edema, and the disposition of thick mucus in the lumen of the bronchi and the bronchioles. The physiological changes which bring about the bronchial asthmatic condition are typically initiated by presence of spasmogens and vasoactivators. Typical of these substances are histamines and some leukotrienes and prostaglandins.

Treatment regimens for chronic asthma today include a wide variety of substances including antihistaminics, cough syrups, bronchodilators, and anti-inflammatory agents. Of this group, bronchodilators and inhaled steroids have proven the most successful. In spite of such success, there are multiple problems associated with the use of inhalers, which are the most widely used type of bronchodilators.

A fundamental problem is poor patient inhaler use training. Successful use of the typical metered dose inhaler (“MDI”) depends on a variety of factors, including position, initial exhalation, spray ejection, and inhalation. Many adults find the coordination required for the series of steps difficult to master, while many children are unable to master the steps at all.

Another problem with the metered dose inhaler is that the great majority of the medication dose “rains out” in the oropharynx and, in fact, most of the dose is swallowed. Only between about 10 to 15 percent of the medication finds its way into a major airway, thus, by way of definition, only a small amount of the medication ends up in the distal airways and alveolar tissue.

An additional problem of inhalers has to do with patient dosing schedules. Schedules can be overly demanding, resulting the patient's failure to adhere to times and intervals. The greater the frequency of doses, the less likely the patient will adhere to the regimen. The individual problems of improper inhaler use and cumbersome dosage schedules result in poor patient regimen adherence.

Anti-inflammatory agents for use in relation to the resolution of asthmatic symptoms are provided as inhaled steroids. These are the most important and effective anti-inflammatory medications available for long-term use. However, these substances suffer from serious undesirable side effects including pituitary-adrenal suppression, osteoporosis, growth rate suppression in children, and, as recently discovered, increased rates in incidences of cataracts and glaucoma. Also, inhaled steroids are slow starters and it may take a week before their positive (or negative)effects become evident. Accordingly, proper administration of these medications includes minimal administration by the lowest effective dose to maintain asthma control.

The other anti-inflammatory agents that have been used include cromolyn sodium, neldocromil, and methylxanthenes. These agents—the cromolyn sodium and the neldocromil substances -are mild to moderate anti-inflammatory agents with excellent safety histories, but embody slow activity and unpredictability in their efficacy the methylxanthenes also demonstrate mild anti-inflammatory effects, but they have a narrow safety margin and are, accordingly, not drugs of choice.

The newer anti-inflammatory agents are leukotriene receptor antagonists and include montelukast sodium and zafirlukast. The leukotrienes are very important mediators in inflammation of the bronchial tubes and are considered to be a very important step in causing inflammation. These medications modify the effects of leukotrienes and result in better control of asthma, night-time symptoms, the mornings with asthma and daytime symptoms. However, these agents are not as effective as inhaled corticosteriods in dealing with asthma. The extent of the burden of bronchial asthma on the national health care system is remarkable, being responsible for almost 15 million outpatient visits, almost half a million 25 hospitalizations per year, and over one million emergency room visits per year. Given the scope of the problem, it is no great wonder that medical costs necessary to respond to the needs of bronchial asthmatics in the United States alone are in the billions of dollars.

A satisfactory treatment regimen for bronchial asthma remains wanting.

### SUMMARY OF THE INVENTION

Accordingly, it is general object of the present invention to provide a composition and method for the treatment of respiratory disease, particularly bronchial asthma.

It is accordingly an object of the present invention to provide a composition and method which is effective, well-tolerated and easily administered, thus being advantageous in the treatment of asthma.

It is a further object of the present invention to provide such a composition and method which utilizes a combination of known medications so as to avoid unnecessary undesirable reactions.

A further object of the present invention is to provide such a composition and method which provides effective and safe resolution of asthma, particularly bronchial asthma. This would be effective for all groups of asthmatics, regardless of the patient's age, sex, physical shape, presence or absence of allergic reaction, or type of asthma (e. g., exercise induced asthma, etc.). Accordingly, a broad range of patients would benefit from the composition of the present invention.

The present invention may also provide benefit for a number of other conditions which are related to asthma. For example, COPD or bronchitis may be relieved by the present invention. The composition of the present invention may even find applicability in any condition in which temporary inflammation of the bronchioles, caused by both bacterial and viral infections (as in the case of “colds”), is present. Specifically, the present composition may be helpful in reducing the duration of the disease as well as related symptoms. In fact, in inflammation of the bronchial tubes associated with both acute and chronic infection, treatment with the present composition may become the drug therapy of choice.

These and other objects of the present invention are achieved by the provision of a treatment composition and regimen which includes a pair of compatible receptor antagonists and an adrenergic bronchodilator. The pair of compatible receptor antagonists include a leukotriene receptor antagonist and a histamine H₁,-receptor antagonist. The adrenergic bronchodilator is a beta₂-adrenergic bronchodilator.

### DETAILED DESCRIPTION OF THE PREFERRED EMBODIMENTS

The present invention mitigates the adverse effects of asthma, particularly bronchial asthma. The present invention accomplishes this step through the provision of a treatment composition and regimen which includes a pair of compatible receptor antagonists and an adrenergic bronchodilator. The pair of compatible receptor antagonists include a leukotriene receptor antagonist and a histamine H₁-receptor antagonist. The adrenergic bronchodilator is a beta₂-adrenergic bronchodilator.

The leukotriene receptor antagonist is taken from the group consisting of montelukast sodium (C₃₅H₃₅ C1NNaO₃S) and zafirlukast (C₃₁H₃₃N₃O₆S).

The histamine II₁-receptor antagonist (antihistamine)is taken from the group consisting of ceterizine hydrochloride ((C₂₁H₂₅C1N₂O₃) (2NaC1)), loratadine (C₂₂H₂₃C1N₂O₂), and fexofenadine (C₃₂H₃₉NO₄) HCL.

The adrenergic bronchodilator is a beta₂-adrenergic bronohodilator is albuterol sulfate ((C₁₃H₂₁NO₃)₂(H₂SO₄)).

### Particular Composition Components

1. The Leukotriene Receptor Antagonists

a. Montelukast Sodium

This is a selective and orally active leukotriene receptor antagonist. It functions by inhibiting certain cysteinyl leukotriene receptors.

### b. Zafirlukast

This is a synthetic selective peptide leukotriene receptor antagonist. It functions by acting as a selective and competitive receptor antagonist of leukotriene.

2. The Histamine H₁-Receptor Antagonists (antihistamines)

### a. Ceterizine Hydrochloride

This is an orally active and selective H₁-receptor antagonist. As an antihistamine, it functions by inhibiting certain peripheral H₁ receptors.

### b. Loratadine

This is a long-acting tricyclic antihistamine. Like ceterizine, it functions by inhibiting certain peripheral H₁ receptors.

### c. Fexofenadine

This is a histamine H₁-receptor antagonist. Again, like ceterizine and loratadine, it functions by inhibiting certain peripheral H₁ receptors.

3. The Adrenergic Bronchodilator Beta₂ Adrenergic Bronchodilator

### a. Albuterol Sulfate

This is a racemic form of albuterol and is a relatively selective beta₂-adrenergic bronchodilator. It functions by having a preferential effect on beta₂-adrenergic receptors.

### TREATMENT REGIMEN AND COMPOSITION EXAMPLE

The composition and method of the present invention provides flexibility of dosage schedule. Preferably, two choices are available to the patient. One dosing schedule allows for a single dose to be taken at nighttime. The other allows for two doses to be taken, one in the morning and one in the evening. The single dosing schedule is suitable both for children and for adults. The twice-daily dosing schedule is suitable for both adults and children over 12 years of age.

1. Once Daily Dose Component

A typical formulation of the single dose component of the present composition includes:

A. For patients aged 12 years and older:

| | | | |
|---|---|---|---|
| 1. | Montelukast Sodium | 10.0 | mg |
| 2. | One of | | |
| | Cetirizine | 5.0 to 10.0 | mg |
| | Loratadine | 5.0 to 10.0 | mg |
| | Fexofenadine | 60.0 to 180.0 | mg |
| 3. | Albuterol | 4.0 to 8.0 | mg |

(In a 4.0 mg tablet, 2.0 mg of Albuterol is in its immediate release form; 2.0 mg is in its extended release form and is double in the 8.0 mg tablet)

B. For patients aged 6 to 11 years:

| | | | |
|---|---|---|---|
| 1. | Montelukast Sodium | 4.0 to 5.0 | mg |
| 2. | One of | | |
| | Cetirizine | 2.5 to 5.0 | mg |
| | Loratadine | 2.5 to 10.0 | mg |
| | Fexofenadine | 30.0 | mg |
| 3. | Albuterol | 2.0 | mg |

(1.0 mg of Albuterol is in its immediate release form; 1.0 mg is in its extended release form)

C. For patients aged 2 to 5 years:

| | | | |
|---|---|---|---|
| 1. | Montelukast Sodium | 4.0 | mg |
| 2. | One of | | |
| | Cetirizine | 2.5 to 5.0 | mg |
| | Loratadine | 2.5 to 10.0 | mg |
| | Fexofenadine | 30.0 | mg |
| 3. | Albuterol | 0.1 | mg/kg |

(0.1 mg/kg of Albuterol is in its immediate release form; 0.1 mg/kg is in its extended release form)

II. Twice Daily Dose Component

A typical formulation of the twice-daily dose component of the present composition includes:

For patients aged 12 years and older:

| | | | |
|---|---|---|---|
| 1. | Zafirlukast | 10.0 to 20 .0 | m |
| 2. | One of | | |
| | Cetirizine | 5.0 | mg |
| | Loratadine | 5.0 | mg |
| | Fexofenadine | 60.0 to 90.0 | mg |
| 3. | Albuterol | 4.0 to 8.0 | mg |

(2.0 mg of Albuterol is in its immediate release form; 2.0 mg is in its extended release form)

The recommendation of the twice-daily dose component for 5 patients aged younger than 12 years is pending.

### Clinical Trials

Clinical trials were undertaken to evaluate the effectiveness of the present composition and treatment regimen. Twenty-five patients known to be asthmatic were administered the composition according to the treatment doses and schedules set forth above. The results were divided according to two categories.

a. Asthma Endpoint Measurements

The asthma endpoints were studied both as primary and secondary endpoints. According to the primary endpoints, or forced expiratory volume 1 (FEVI) which are daytime endpoints, the asthmatic patients showed marked improvement in both FEV₁ and daytime asthma symptoms. According to the secondary endpoints, or nighttime peak expiratory flow rate (AM PEFR), the asthmatic patients demonstrated marked improvement in this category and did not suffer as much with nocturnal awakenings due to asthmatic episodes compared with previous nights without treatment using the composition of the present invention. The use of beta-agonist inhalers was significantly reduced in both the number of inhalations used daily and the percentage of days when the inhaler was actually used.

b. Asthma Outcome Measurements

The asthma outcome was studied from five different perspectives.

I. Asthma exacerbations were fewer and further between as compared with non-treatment with the present composition.

II. Asthmatic attacks were reduced significantly as compared with non-treatment with the present composition.

III. Global asthma evaluation, which evaluated how many patients experienced improved feelings from the patient's perspective and as expressed by a treating physician's evaluation, showed that most of the patients experienced improved feelings as compared with non-treatment with the present composition.

IV. Quality of life evaluation of the patients measuring daytime symptoms and asthma aggravated by exercise and under increased emotional stress demonstrated marked improvement as compared with non-treatment with the present composition.

V. Exercise induced bronchial asthma was significantly improved as compared with non-treatment with the present composition.

Those skilled in the art can now appreciate from the foregoing description that the broad teachings of the present invention can be implemented in a variety of forms. Therefore, while this invention has been described in connection with particular examples thereof, the true scope of the invention should not be so limited since other modifications will become apparent to the skilled practitioner upon a study of the drawings, specification and following claims.

What is claimed is:

1. A composition for the treatment of asthma, the composition comprising:
a first receptor antagonist and a second receptor antagonist, said first and second receptor antagonists being selected from the group consisting of leukotriene receptor antagonists and histamine receptor antagonists; and
an adrenergic bronchodilator.

2. The composition of claim 1, wherein said adrenergic bronchodilator is a beta₂-adrenergic bronchodilator.

3. The composition of claim 2, wherein said beta₂-adrenergic bronchodilator is albuterol sulfate.

4. The composition of claim 1, wherein said leukotriene receptor antagonist is selected from the group consisting of montelukast sodium and zafirlukast sodium.

5. The composition of claim 1, wherein said histamine receptor is a histamine H₁-receptor antagonist.

6. The composition of claim 5, wherein said histamine H₁-receptor antagonist is selected from the group consisting of ceterizine hydrochloride, loratadine, and fexofenadine.

7. A composition for treatment of asthma, the composition comprising:
a first receptor antagonist having a first chemical composition;
a second receptor antagonist having a second chemical composition, said first chemical composition and said second chemical composition being chemically dissimilar; and
an adrenergic bronchodilator.

8. The composition of claim 7, wherein said first receptor antagonist and said second receptor antagonist are selected from the group consisting of leukotriene receptor antagonists and histamine receptor antagonists.

9. The composition of claim 8, wherein said andrenergic bronchodilator is a beta₂-adrenergic bronchodilator.

10. The composition of claim 9, wherein said beta₂-adrenergic bronchodilator is albuterol sulfate.

11. The composition of claim 8, wherein said leukotriene receptor antagonist is selected from the group consisting of montelukist sodium and zafirlukast sodium.

12. The composition of claim 8, wherein said histamine receptor is a histamine H₁-receptor antagonist.

13. The composition of claim 12, wherein said histamine H₁-receptor antagonist is selected from the group of consisting ceterizine hydrochloride, loratadine, and fexofenadine.

14. A composition for the treatment of asthma, the composition comprising:
montelkast sodium;
an antihistamine selected from the group consisting of cetirizine, loratadine, and fexofenadine; and
a sympathomimetic bronchodilator.

15. The composition of claim 14, wherein said sympathomimetic bronchodilator is albuterol.

16. A method for treating asthma comprising the steps of:
preparing a composition comprising:
a first receptor antagonist in the amount of between about 4.0 mg and about 20.0 mg;
a second receptor antagonist in the amount of between about 2.5 mg and about 180.0 mg, said second receptor antagonist being different from said first receptor antagonist;
an adrenergic bronchodilator in the amount of between about 4.0 mg and about 8.0 mg; and
administering said composition to a patient.

17. The method of claim 16, wherein said first receptor antagonist and said second receptor antagonist are selected from the group consisting of leukotriene receptor antagonists and histamine receptor antagonists.

18. The method of claim 16, wherein said adrenergic bronchodilator is a beta₂-adrenergic bronchodilator.

19. The composition of claim 18, wherein said beta₂-adrenergic bronchodilator is albuterol sulfate.

20. The composition of claim 17, wherein said leukotriene receptor antagonist is selected from the group consisting of montelukast sodium and zafirlukast sodium.

21. The composition of claim 17, wherein said histamine receptor is a histamine H₁-receptor antagonist.

22. The composition of claim 21, wherein said histamine H₁-receptor antagonist is selected from the group consisting of ceterizine hydrochloride, loratadine, and fexofenadine.
